(19) United States
(12) Patent Application Publication
(10) Pub. No.: US 2018/0317772 A1
(43) Pub. Date: Nov. 8, 2018

(54) DEVICE FOR BIOCHEMICAL MEASUREMENTS OF VESSELS AND FOR VOLUMETRIC ANALYSIS OF LIMBS

(71) Applicants: UNIVERSITE DE MONTPELLIER, Montpellier (FR); CENTRE NATIONAL DE LA RECHERCHE SCIENTIFIQUE, Paris (FR); AXLR SATT Du LANGUEDOC ROUSSILLON, Montpellier (FR); CENTRE HOSPITALIER UNIVERSITAIRE de MONTPELLIER, Montpellier (FR); CENTRE HOSPITALIER UNIVERSITAIRE de NÎMES, Nîmes (FR)

(72) Inventors: Jean TRIBOULET, Nîmes (FR); Michel DAUZAT, Nîmes (FR); Florent VEYE, Grabels (FR); Sandrine MESTRE, Montpellier (FR); Isabelle QUERE, Nîmes (FR); Nicolas BERRON, Cournonsec (FR)

(21) Appl. No.: 15/740,744

(22) PCT Filed: Jul. 1, 2016
(86) PCT No.: PCT/EP2016/065559

§ 371 Date: Dec. 28, 2017

(30) Foreign Application Priority Data
Jul. 3, 2015 (FR) 1556324

(51) Int. Cl. A61B 5/00; A61B 5/107; A61B 8/00; A61B 8/08; A61B 5/02

(52) U.S. Cl. CPC A61B 5/0035; A61B 5/1073; A61B 8/4416; A61B 8/485; A61B 5/0064; A61B 5/1079; A61B 5/0053; A61B 8/40; A61B 5/02007; A61B 8/488; A61B 5/004

(57) ABSTRACT

A non-wounding system for bio-morphological characterization of a human limb including a device for geometric and volumetric, data, including:
a plurality of systems for three-dimensional images acquisition for imaging the limb,
an articulated and motorized frame arranged for positioning some of the systems for acquisition in a peripheral manner with respect to the limb,
a device for processing the data, arranged for representing the data in the form of a points presenting a set of three-dimensional coordinates,
a device for biomechanical measurements including a probe holder fixing at least one ultrasound probe for imaging the vascular system relative to the limb and a force sensor measuring the pressure exerted by said probe on the limb, and
a device for analysis, for merging the volumetric data and some of the anatomical and biomechanical data, and also for determining morphological variables and/or biomechanical variables of the vascular system of the limb.

### TECHNICAL FIELD

The present invention relates to a non-wounding device for bio-morphological characterization of a human limb and biomechanical assessment of the blood vessels, as well as a method for assisting the definition of compression orthosis adapted to the limb to be treated.

The invention is related to the field of medical instrumentation.

### STATE OF THE ART

Although very widely used in numerous cases, such as the treatment of chronic venous insufficiency and lymphoedema, compression orthoses are not always perfectly adapted either to the morphology of the limb on which they are put in place, or to the biomechanical characteristics of the veins, their wall, and their tissue environment. In fact, compression orthoses are manufactured based on standards relating to a morphological model, without taking the individual, in particular morphological, characteristics of each patient into account. The therapeutic result is thus not always optimally achieved.

Several morphological characterization and volume measurement techniques are known and applicable in the medical field:
A first technique consists of measuring the volume of water displaced during the immersion of the limb to be treated. This technique, although theoretically simple, is not always easy to implement depending on the degree of mobility and/or state of health of the patient, for example after recent surgery or in the case of skin lesions. Moreover, it does not make it possible to carry out local or segmental measurements, and it therefore does not make it possible to reveal the distribution of oedema in the limb.
Another technique consists of carrying out perimeter measurements at different levels of the limb. This technique is very simple to implement and widely practised in the hospital environment; however, it is very approximate, tedious and poorly reproducible.
Other techniques use various methods, such as infrared light beams, for carrying out measurements of diameters in stages along the limb and to reconstruct its silhouette (generally in two planes), but these measurements remain approximate, in particular in the case of deformations linked to oedema, and do not accurately indicate the volume of the extremity (hand or foot).
Finally, 3D volumetric laser scanning is already used in the medical field for the morphological characterization of parts of the body (face, limb etc.) but has not been the subject of technical developments specific to medical use (particularly in terms of ergonomics or rapidity of acquisition), nor of utilization for customizing orthoses as a function of the morphological and biomechanical characteristics of the veins of the patient.

Moreover, though numerous studies have been conducted to investigate the effects of venous compression, none has made it possible to assess in real time the biomechanical characteristics of the vein wall as well as the stresses experienced and transmitted by the tissues of a limb on which a compression orthosis has to be put in place.

An object of the present invention is to at least substantially respond to the above problems while offering other advantages.

Another purpose of the invention is to solve at least one of these problems by means of a novel system of volumetric measurements and morphological characterization of a human limb, in parallel with the anatomical and biomechanical assessment of the blood vessels of the limb and, if appropriate, to relate them to the compression parameters.

Another purpose of the invention is to propose such a system of ergonomics that is adapted for medical use, easy to implement, non-invasive and non-wounding. Another purpose of the invention is to propose such a system that is capable of carrying out reproducible, reliable and accurate measurements.

### DISCLOSURE OF THE INVENTION

At least one of the abovementioned objectives is achieved with a non-wounding system for bio-morphological characterization of a human limb comprising:
a device for geometrical—preferably three-dimensional (3D)—and volumetric measurements comprising:
   a plurality of systems for three-dimensional images acquisition arranged for imaging said limb,
   an articulated and motorized frame arranged both for positioning at least some of the plurality of the systems for acquisition in a peripheral manner with respect to said limb, and also for moving at least some of the plurality of the systems for acquisition with respect to said limb,
   a device for processing the geometric and/or volumetric data, arranged for representing the acquisition data in the form of a plurality of points presenting a set of coordinates in a three-dimensional frame of reference, for example, in the form of a mesh optionally including textured information on the limb observed and provided by the systems for acquisition of images, and
a device for anatomical and biomechanical measurements comprising a probe holder comprising:
   an ultrasound probe for imaging the vascular system relating to said limb, and
   a force sensor arranged for measuring the pressure exerted by said probe on the limb, said force sensor being fixed to said ultrasound probe.

Thus, the system for characterization according to the invention makes it possible to propose a new device for characterizing a limb, in which the device for measurement and for volume measurement to reconstruct a 3D digital model of said limb is supplemented by a device for anatomical and biomechanical measurements making it possible to determine a certain number of morphological and/or biomechanical variables of the afferent vascular system of said limb. It is thus possible to measure and understand, among other things, the role of the mechanical stresses on the vascular walls in the vascular disease affecting said limb, and to foresee the effects of the different compression means and forces in order to fine-tune the prescription thereof.

By way of non-limitative examples, the invention, for the anatomical and biomechanical measurements, aims to characterize, both the anatomy and the geometry—in particular diameter, circumference, section, parietal thickness—and on the other hand the biomechanical characteristics—such as the modulus of elasticity—of the blood vessels in order to better adapt the compression orthosis to said limb.

The anatomical and biomechanical measurements are mainly, but not exclusively, carried out by an ultrasound system coupled with a measurement of the force exerted by the ultrasound probe on said limb during the measurement. The coupling of the ultrasound probe and the force sensor is carried out by the probe holder. This clever coupling and integrated in an innovative manner into a device for characterizing a human limb makes it possible both to control the pressure exerted by the operator and to record the force exerted in return on said ultrasound probe, representative of the interstitial pressure as well as the local blood pressure and variations thereof.

The ultrasound measurements advantageously make it possible to determine in particular the dimensional and/or transverse properties of said blood vessels. More particularly, the position and orientation of the cross-section of the blood vessel is determined, optionally in different positions.

The measurement of force concomitant with the ultrasound measurement can also be used in order to standardize the biomechanical measurements thus carried out, for example using an automatic control making it possible to maintain the force, pressure, or the effects thereof at a set level.

The system for characterization according to the invention thus makes it possible to measure the vascular and/or arterial variables without inserting sensors and/or medical devices inside said limb, thus helping to improve the ergonomics of the measuring system and the comfort of the patient, while excluding the various risks (in particular of haemorrhage or infection) associated with the wounding techniques.

According to a particular embodiment, the ultrasound imaging is carried out in real time in order to be able to study the dynamic evolution of the morphological and biomechanical variables of the vascular system, such as for example the variation in the arterial diameter during the cardiac cycles.

It is thus possible to assess in real time the effect of the different compression means and parameters on the geometry of the superficial and deep vessels.

The 2D geometry and volume measurements of the limb to be characterized are carried out by means of a plurality of three-dimensional sensors placed around said limb.

The number of three-dimensional sensors can vary as a function of the size and shape of said limb, the degrees of freedom of the frame on which they are mounted, as well as the intrinsic characteristics of said three-dimensional sensors (resolution, field covered, scope etc.) and the scanning time constraints.

In order to carry out complete or partial scanning of the limb to be characterized, it is necessary for the surface of said area to be scanned to be completely imaged by the plurality of three-dimensional sensors. Thus, if only a segmental part of said limb has to be imaged, the plurality of three-dimensional sensors utilized has to be arranged so as to at least collectively scan the entire surface of said segmental part. If all of the limb has to be imaged, the plurality of three-dimensional sensors utilized then has to be arranged so as to at least collectively scan the entire surface of said limb. The number and arrangement of the three-dimensional sensors utilized can be adapted depending on the situation.

The complete scanning of the segmental part or of the entire limb can be achieved by any means and can thus comprise means, optionally motorized, for moving said three-dimensional sensors around the limb to be characterized if the field of measurement of the three-dimensional sensors does not make it possible to image all of the surface from a single position or in order to shorten the scanning time.

In the case where a movement of said sensors around said limb is necessary, it can be carried out by the articulated frame, which has at least one means arranged both for supporting at least one three-dimensional sensor and for carrying out a movement relative to said limb.

This movement can be predefined via at least one particular kinematic connection. It can for example be a rotational movement and/or a translational movement. In a general manner, the at least one means is arranged in order to allow said at least one three-dimensional sensor to measure at least one other part of the surface of the limb to be characterized or of the segmental part of said limb.

Advantageously, the articulated frame can be motorized in order to more finely control said movements of the sensors with respect to the limb to be measured.

Preferentially, the means for motorization of said frame can be arranged to be remote-controlled in order to program particular and/or predefined movements.

The three-dimensional sensors can be of any type, and are designed in order to produce a volume mesh of the imaged surface.

Preferentially, the device according to the invention utilizes a plurality of three-dimensional laser cameras, advantageously seven.

By way of non-limitative example, each three-dimensional sensor thus independently produces a mesh of said surface or of the segmental part of said limb. Each three-dimensional sensor scans the surface of at least a part of said limb in the form of a set of points having a particular set of coordinates in a particular three-dimensional frame of reference.

In order to be able to reconstruct a complete volume mesh of at least a part of said limb, the device according to the invention utilizes means for processing the measurement data, which are arranged in order to aggregate the different sets of points of the different sensors in a single three-dimensional frame of reference.

Alternatively, at least one three-dimensional sensor can be used for recording the successive positions of at least some of the other three-dimensional sensors, said at least one sensor used to record their respective positions being able to be immobile and/or at least one predetermined position.

Moreover, advantageously, each three-dimensional sensor is calibrated and/or has intrinsic calibrating means which make it possible to make the three-dimensional frames of reference of each set of points compatible.

Advantageously and/or alternatively, the system for morphological characterization utilizes means for calibration common to at least some of the three-dimensional sensors in order to make the three-dimensional frames of reference of said at least some of the three-dimensional sensors compatible and/or identical.

The device for geometric and volumetric measurements thus makes it possible to scan at least a part of the limb to be characterized rapidly and accurately. In fact, by using a plurality of three-dimensional sensors optionally mobile around said limb, the times for acquisition of the images are reduced since each sensor is only responsible for measuring at least a part of said limb. The volume mesh thus obtained is more accurate as the measurement thus carried out is more comfortable for the patient, more rapid and thus less susceptible to an unwanted movement of the limb during recording, due to the patient's discomfort.

The device for characterization is thus more ergonomic since, during this scanning phase, it meets a need to improve comfort at the same time.

The measurements carried out with the system for characterization according to the invention can be carried out equally well in the presence or in the absence of the orthosis in order to accurately measure the effects thereof on at least a part of the limb.

Moreover, the anatomical and biomechanical measurements can be carried out at the same time as the volumetric measurements or alternately.

According to a particular embodiment, the system according to the invention can also comprise a device for analysis, arranged both for merging at least a part of the volumetric data and at least a part of the anatomical and biomechanical data, and also for determining morphological variables of said limb and/or biomechanical variables of the vascular system of said limb.

Data merging consists of a set of processes aimed at integrating multiple data, representing a varied number of different physical measurements (for example optical, mechanical, electric etc.) of the same object, in order to aggregate them in a single, coherent, accurate and useful representation.

By way of non-limitative example, data merging can for example consist of superimposing the ultrasound measurements—and the morphological variables of the vascular system thus characterized—on the digital volume model of the limb in order to visualize a digital representation that is faithful to the reality of the vascular system during at least one cardiac cycle or a dynamic manoeuvre (movement, compression etc.) and its location in said limb.

The device for analysis according to the invention thus makes it possible to aggregate at least some of the volumetric data and at least some of the biomechanical data in order, in particular, to establish relationships between the biometric data measured by the device for biomechanical measurements and the digital model of the at least one part of the limb.

In order to do this, the device for analysis can implement the following analysis method, for example:
determination of the influence of the orthosis on at least one part of the vascular system, and more particularly on the variation in its cross-section,
coupling of these results with the measurements of the dimensional variations of the limb,
determination of the blood flow conditions in the at least one part of the vascular system, for example in the presence of pathology (insufficiency, stenosis, thrombosis etc.), in order to understand the different forces involved,
integration of these data on the digital model of the at least one part of the limb.

The blood flow conditions in the vascular system are determined using at least one representative variable, preferentially of the digital type. This variable is deduced/calculated from the different measurements carried out. It is then merged with the geometric model in order to visualize, on a three-dimensional digital representation, the distribution of said representative variable of the vascular system of the limb.

Data merging thus makes it possible to superimpose dimensional, optionally dynamic, measurements, with surface or deep biomechanical measurements carried out on the at least one part of the limb in order to accurately locate said biomechanical measurements and to improve understanding of the effects of the orthosis on said limb.

Advantageously, at least some of the plurality of systems for three-dimensional images acquisition of the system according to the invention can operate synchronously.

It is thus possible to reduce the scanning time of the at least a part of the limb to be characterized.

Preferentially, in the system according to the invention, the device for volumetric measurements can also comprise a tool assisting the geometrical and volumetric measurement of said limb, arranged for determining representative areas of said limb for the determination of its shape and its volume.

The representative areas are those which can make it possible to better understand a given pathology affecting said limb and/or be situated around a manifestation or consequence of the pathology. The tool assisting the volumetric measurement can in particular be arranged for detecting particular volumes on a limb, such as for example deformations representative of certain pathologies. By way of non-limitative example, the tool assisting the volumetric measurement can compare the morphology of said limb with a database comprising typical morphologies of said limbs, as described in the standards.

In a particular version of the system according to the invention, the frame can comprise at least one arm for supporting at least some of the plurality of systems for three-dimensional images acquisition.

Optionally, said at least one arm is arranged for pivoting about said limb.

Advantageously, the amplitude of rotation of the at least one arm of said frame can be comprised between 0 and 90° . The amplitude of the rotation of the arms of the frame and supporting at least some of the three-dimensional sensors is, as described above, determined in particular by the need to achieve a covering of the surfaces of the limb to be characterized between at least some of the three-dimensional sensors and at least others. Typically, the necessary amplitude of rotation is of the order of approximately fifteen degrees.

According to a preferential version of the invention, the probe holder can be mounted on an articulated and/or motorized arm fixed, or not fixed, to the frame, and arranged for bringing said probe holder into contact with the limb and/or for moving said probe holder on said limb.

The articulated arm makes it possible to carry out movements in space while supporting the probe holder, thus making it possible to carry out a more accurate examination of the limb to be characterized.

According to an embodiment of this preferential version, the articulated arm can be motorized in order to carry out movements automatically and/or in a predefined manner.

Advantageously, an automatic control of the ultrasound probe in contact with the limb to be characterized as a function of the pressure measured by the force sensor can make it possible to carry out more reliable and more reproducible measurements.

On the other hand, the probe holder has a shape and proportions that make it easy to grasp. It is in particular designed in light materials in order to minimize its weight and facilitate handling of the probe during the characterization of the limb examined. The choice of materials can also be determined by the medical nature of its application: it can preferentially be designed in plastic material.

Advantageously, the device for biomechanical measurements of the system according to the invention can comprise at least one sensor for measuring the interface pressure, placed in contact with the skin of said limb.

Optionally, the device can also be supplemented by an intramuscular pressure sensor for measuring the blood pressure inside a muscle of said limb, and/or an intravascular pressure sensor for measuring the blood pressure inside a vessel of said limb.

According to a particular embodiment of the device for biomechanical measurements according to the invention, the acquisition of the data originating from at least some of the sensors comprised by said device for biomechanical measurements can be carried out synchronously.

The adaptation of the device for biomechanical measurements to the assessment of the vascular physiopathology of at least a part of the limb to be characterized makes it possible, using the device for analysis according to the invention, to merge a larger number of data originating from other sensors preferentially placed on the surface of at least a part of said limb, and making it possible to measure other physical values and/or other morphological, physical or chemical variables. It is thus possible to better understand the effects of the orthosis on said limb.

The device for analysis according to the invention can thus also make it possible to relate the variations in the interface pressure to, for example, both the intramuscular or interstitial pressure and blood pressure, and also the geometry of the different vessels examined, superficial and deep.

The interface pressure can be measured by different types of sensors, preferentially hydraulic or pneumatic, by displacement of a fluid inside a pouch or a flat cuff in contact with the skin.

Electrical sensors (resistive or capacitive) are also known.

The interface pressure sensors are distributed on the surface of the limb to be characterized, preferentially according to a standardization well known to a person skilled in the art.

Preferentially, the interface sensors are arranged to be brought into contact with the limb to be characterized, in the presence or in the absence of the compression orthosis.

By way of non-limitative example, the interface pressure sensors can consist of pneumatic sensors associated with piezoelectric pressure transducers.

The acquisition of the data originating from the different sensors used for the biomechanical measurements and/or from the plurality of three-dimensional sensors used for the volumetric measurements is carried out by any known means, in analogue and/or digital manner. Finally, the data are all digitized in order to be utilized by a processing unit, preferentially a computer.

Optionally, a means for conditioning, shaping and/or pre-processing the signals originating from the at least one of the different sensors comprised within the device for biomechanical measurements can be utilized in the system for characterization according to the invention.

Typically, but non-limitatively, the device according to the invention thus measures at least one mechanical property of the superficial and/or deep vascular system, in order, as explained previously, to determine a representative digital parameter and merge it with the three-dimensional geometric model.

Advantageously, the measured mechanical property is the compression of said vascular system under the effect of the application of the probe thereto, and measured by the force sensor borne by the probe holder.

The measurement is carried out at one or more points and over a period making it possible to measure its evolution in time, as a function, for example, of the pressing and removal of the probe. This measurement thus makes it possible to measure the compression and expansion of the vascular system under the effect of this exerted pressure.

The representative variable calculated from these measurements is the elasticity of the wall of the vascular system, making it possible to show the distensibility and/or the compliance of the corresponding vascular wall.

This representative variable is deduced from the measurement and ultrasound image produced, and then calculated according to several known means, including modelling.

By way of non-limitative example, a model based on assessment of the hysteresis observed based on changes in the vascular wall during the compression and expansion of the vascular system ultimately makes it possible to calculate the elasticity of the vascular system.

According to another aspect of the same invention, a method is proposed, for assisting the definition or the selection or the adaptation of compression orthoses for a limb, implementing the system for bio-morphological characterization according to any one of the embodiments of the invention, comprising at least one of the following steps:
geometric and volumetric measurements of said limb,
biomechanical measurements of said limb,
merging of the geometric and/or volumetric and biomechanical measurements in order to correlate at least some of the geometric and/or volumetric measurements and at least some of said biomechanical measurements.
determining at least one biometric and/or at least one volumetric variable.

The method according to this other aspect of the invention also makes it possible to adapt a pre-existing orthosis to the geometry of the limb on which it was used.

According to a preferred embodiment of this aspect of the invention, the biomechanical measurements can be carried out at least during the step of geometric and/or volumetric measurements of said limb.

As explained in the previous paragraphs, the biomechanical data are used in order to determine a certain number of morphological and/or biomechanical variables representative of the vascular system of said limb. These measurements can be carried out dynamically.

The morphological and/or biomechanical variables representative of the vascular system are mainly deduced from the ultrasound images and then supplemented by the measurements from at least one other sensor.

By way of non-limitative example, a model based on assessment of the hysteresis observed in changes in the vascular wall imaged by the ultrasound probe during the compression and expansion of the vascular system ultimately makes it possible to calculate the elasticity of the vascular system.

Advantageously, the biomechanical measurements are carried out at a single point, thus making it possible to measure a variable representative of the vascular system at this point. The representative variable is then propagated to the entire vascular system, assuming that the biomechanical properties of said vascular system are isotropic and homogeneous.

Alternatively, a mathematical model can propagate the value of said representative variable through the digital model of said vascular system in order to calculate estimated values of said representative variable as a function of the value measured and calculated at a point.

Alternatively, the measurements are carried out at several points and/or in several different areas in order to fine-tune said mathematical model and to calculate several values of the representative variable as a function of the location of the portion of the vascular system in question.

According to a particular embodiment, the method according to the invention can comprise a step of pre-processing the ultrasound images produced, before merging the data. This pre-processing step consists, in particular, of processing the noise of the images and/or removing or identifying the artefacts (diffraction, refraction, inclusions etc.) in order to facilitate the extraction of the geometric information.

A subsequent step consists, moreover, of extracting the contours of at least a part of at least one recorded ultrasound image. In order to do this, several methods well known to a person skilled in the art exist, such as derivative methods, methods based on segmentation, active contours etc.

These pre-processing methods can be carried out once all the measurements have been carried out—post-processing—or carried out in real time as the different data are acquired. In all these cases, they make it possible to bring the data obtained by said measurements into alignment.

On the basis of the results of data merging and/or as a function of the results of volumetric and biomechanical measurements, and according to an advantageous version of this aspect of the invention, the method can also comprise a step of defining, adapting or selecting a compression orthosis for the limb, as a function of the at least one biometric variable and/or the at least one geometric and/or volumetric variable.

It can also preferentially comprise an additional step of developing a biomechanical model predicting the effects of the compression orthosis on the limb and its vascular system.

### DESCRIPTION OF THE FIGURES AND EMBODIMENTS

Other advantages and characteristics of the invention will become apparent through the following description of several embodiments given by way of indicative and non-limitative examples, and from the attached diagrammatic drawings, in which:

FIG. 1A shows an overall diagrammatic view of the device for volumetric measurement according to the invention,

FIG. 1B shows a first embodiment of the device for volumetric measurement according to the invention,

FIG. 2 shows the probe holder used for carrying out some of the biomechanical measurements of the vascular system of the limb,

FIG. 3 shows an articulated arm for the probe holder, according to a particular embodiment of the invention,

FIG. 4 shows the principle of bio-morphological characterization according to the invention, and

FIG. 5 shows an analysis sequence of ultrasound images produced during the biomechanical measurements.

The embodiments which will be described hereinafter are in no way limitative; it is possible, in particular, to imagine variants of the invention comprising only a selection of characteristics described hereinafter, in isolation from the other characteristics described, if this selection of characteristics is sufficient to confer a technical advantage or to differentiate the invention with respect to the state of the prior art. This selection comprises at least one, preferably functional, characteristic without structural details, or with only a part of the structural details if this part alone is sufficient to confer a technical advantage or to differentiate the invention with respect to the state of the art.

In particular, all the variants and all the embodiments described can be combined together if there is no objection to this combination from a technical point of view.

In the figures, the components common to several figures retain the same reference number.

An orthosis is an appliance which compensates for an absent or deficient function of a limb, assists a joint or muscle structure, stabilizes a body segment during a phase of rehabilitation or rest. It differs from a prosthesis, the function of which is to replace a missing part of the human body.

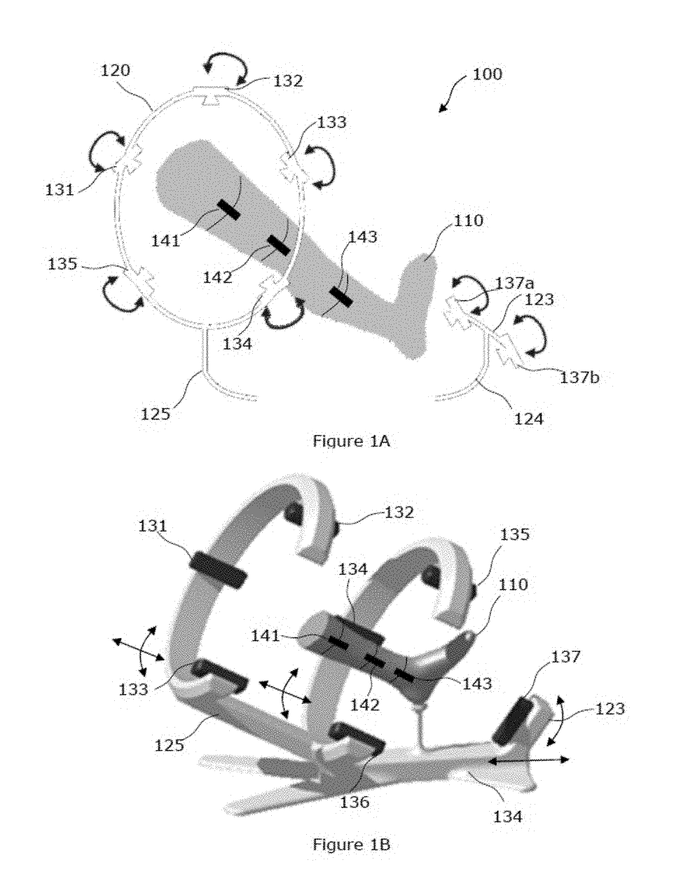

FIG. 1A shows an overall diagrammatic view of the device for volumetric measurement 100 according to the invention, and FIG. 1B shows a particular embodiment of the invention.

The patient, one of whose limbs is to benefit from the application of an orthosis, is positioned on a measurement bench, a part of which is shown in FIG. 1B. The measurement bench typically comprises a first structure—optional and not shown—allowing the patient to be comfortably positioned for the morphological analysis of the limb on which the orthosis is to be put in place, as well as a second structure 100 shown in FIGS. 1A and 1B, making it possible to place said limb 110 inside a measurement area.

More particularly, FIG. 1A diagrammatically shows such an setup for characterizing a lower limb 110.

The lower limb 110 is placed inside an articulated frame 120 which has several three-dimensional sensors 131-137 in the space peripheral to said limb 110. The frame 120 is constituted by a base 124 at the end 123 of which two sensors 137a, 137b make it possible to image the arch of the foot of the limb 110. As an extension with respect to the base 124, a support 125 extends in a direction substantially parallel to the elongation of the lower limb 110.

According to a particular embodiment of the invention, the support 125 supports a circular arm 121, to which the three-dimensional sensors 131-135 are fixed.

The circular arm 121 is articulated in order to allow release to the right or to the left and thus allow the patient to introduce their limb 110 into, or remove it from, the measurement area inside said frame 120.

According to a particular embodiment of the invention, compatible with any version of the frame 120, the support 125 can be telescopic, in order to adapt to the sizes of the lower limbs of different patients.

In FIG. 1A, the circular arm 121 supports five three-dimensional sensors 131-135 which can be articulated and/or motorized so as to carry out a scan around the lower limb 110.

Optionally, the circular arms 121, 122 can also, or alternatively, be articulated and/or motorized so as to carry out a rotation around the lower limb 110.

In other words, the articulation of the different sensors can be collective, i.e. implemented by the articulation and/or the rotation of the arm or arms supporting them and/or of the support; alternatively, the articulation of the different sensors can be individual, each sensor having its own means for articulation and/or rotation with respect to the support or the frame supporting it.

The means for articulation and/or rotation are well known per se, and not described here.

The distance separating the circular arm 121 from the base 124 can also be adjustable so as to adapt the volumetric measurement device 100 to the dimensions of the limb 110 to be characterized.

In addition to the volumetric measurement device 100, FIG. 1A also shows the putting in place of surface pressure sensors 141-143 used for measuring, for example, the pressure exerted by the orthosis on the lower limb 110 when the orthosis is put in place, or the surface pressure in the absence of the orthosis. In FIG. 1A, three sensors 141-143 are thus arranged along the lower limb 110.

Preferentially, the position of the surface pressure sensors 141-143 can be chosen so as to characterize the areas which are also imaged by the three-dimensional sensors 131-137 in order to be able—ultimately—to merge the data and establish a more complete analysis of said limb 110 and of the effect of the orthosis.

In the particular embodiment shown in FIG. 1B, the lower limb 110 is placed inside an articulated frame 120 which has several three-dimensional sensors 131-137 in the space peripheral to said limb 110. The frame 120 is constituted by a base 124 at the end 123 of which a first sensor 137 makes it possible to image the arch of the foot of the limb 110. As an extension with respect to said base 124, a support 125 extends in a direction substantially parallel to the elongation of the lower limb 110 and supports two circular arms 121, 122, to which the three-dimensional sensors 131-136 are fixed.

In this example, each circular arm 121, 122 is arranged, both for allowing easy insertion of the limb 110 to be characterized inside the device 100 and also is articulated so as to move the three-dimensional sensors 131-136 around said limb.

As explained previously, the movement of the three-dimensional sensors 131-136 around said limb can be collective, using motorization and an independent articulation of each arm and/or by an independent articulation and motorization of each sensor in order to allow the latter—collectively and/or individually—to image several areas of the limb.

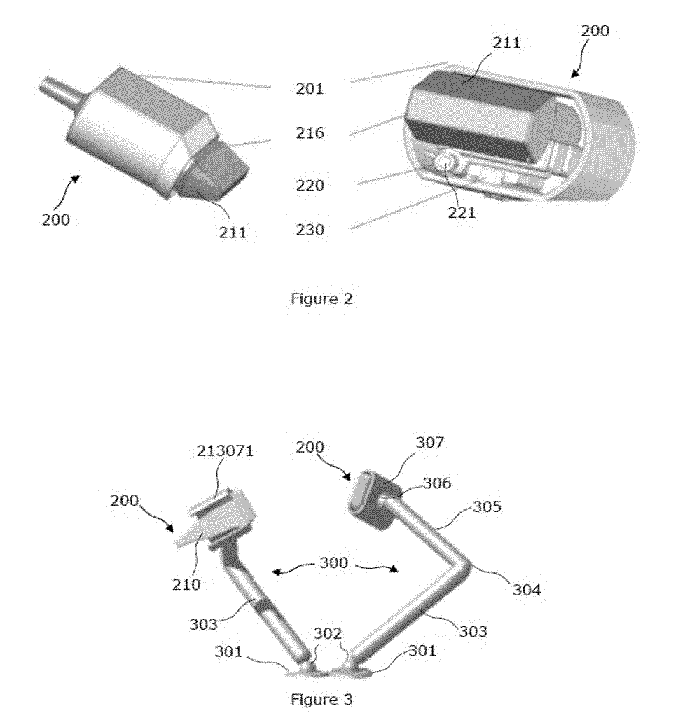

FIG. 2 shows the probe holder 200 used for carrying out some of the biomechanical measurements of the vascular system of the limb 110.

The probe holder is constituted by a housing 201 inside which, or onto which, an ultrasound probe 210 is fixed, mounted on a linear translation support and connected to a force sensor 220. The probe holder 200 is designed so as to allow the insertion of several types of ultrasound probes 210. It thus comprises means for fixing said probe, not shown in FIG. 2, such as for example at least one ring passing through the housing 201 and around the probe 210. The active end of the ultrasound probe 210 projects beyond the probe holder in order to be able to be brought into contact with the skin of the limb 110 to be characterized.

The force sensor 220 is fixed clos to the ultrasound probe 230 by any fixing means 230, and in such a way that it is in contact with the skin of the limb 110 when the ultrasound probe 210 is.

The most significant force measurements are those carried out in the axis of the ultrasound probe 210, i.e. substantially parallel to the active surface 211 of said probe 210. However, additional measurements of forces in the transverse directions can make it possible to fine-tune the measurements and correct certain possible errors linked to a defect of alignment of the force sensor 220 with respect to said ultrasound probe 210.

The force sensor 220 is arranged for measuring at least the force normal to its contact surface 221.

FIG. 3 shows an articulated arm 300 for the probe holder 200 according to a particular embodiment of the invention.

The probe holder 200 is fixed onto an articulated arm 300 using fixing means 307. The articulated arm 300 can be independent of the volumetric measurements device 100, or fixed to said volumetric measurements device 100.

At the end of the articulated arm 300, a ball joint 306 allows the probe holder 200 to carry out three rotations.

At the base 301 of the articulated arm 300, a ball joint 302 makes it possible to orientate the latter in any direction.

Between the two ends, the articulated arm 300 can comprise an unlimited number of kinematic links. In the example shown in FIG. 3, the articulated arm is composed of two intermediate segments 303, 305, linked to each other by a ball joint 304.

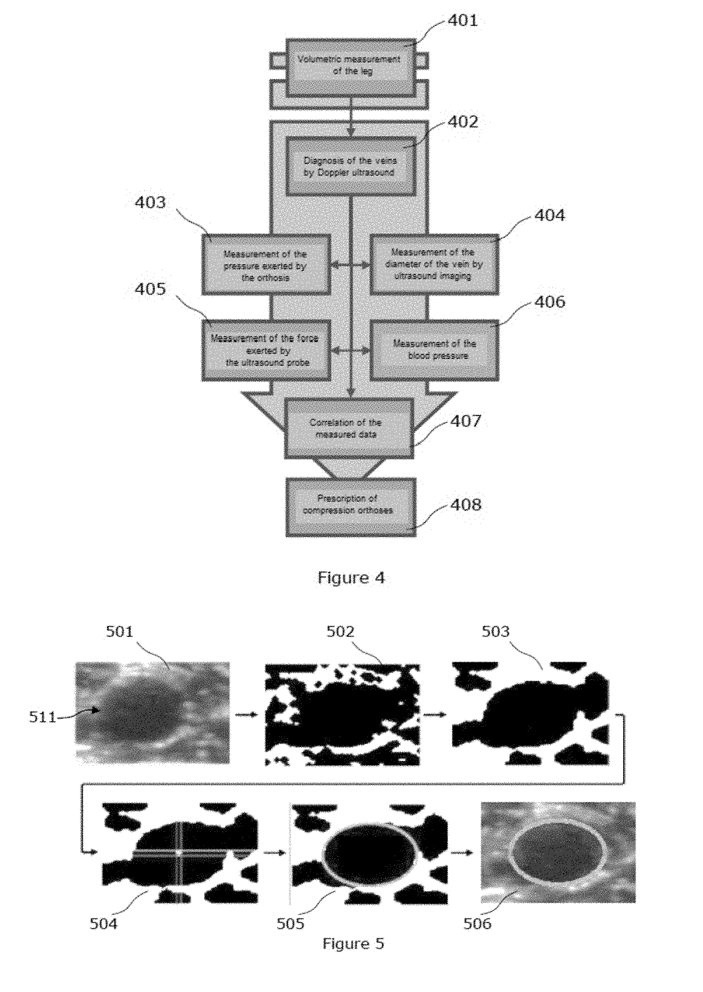

FIG. 4 shows the principle of bio-morphological characterization according to the invention, and comprises the following steps:
the patient is positioned on the analysis bench, and their limb 110 is placed inside the frame 120 supporting the sensors 131-137. Optionally, the limb 110 on which the measurements are to be carried out can be held by a temporary retention device;
in step 401, the volumetric measurements are carried out. The frame 120 moves the three-dimensional sensors 131-137 in order to scan at least a part of said limb 110;
in step 402, at least one ultrasound measurement of at least a part of the vascular system of said limb is carried out using the probe holder 200, and more particularly via the ultrasound probe 210, in order to determine certain morphological variables of said vascular system, in particular the diameter of the vessel in step 404;
in step 405, the development in the force exerted by the probe 210 on the limb 110 during the ultrasound measurements 402 is recorded via the force sensor 220 arranged on the probe holder 200.
in step 403, measurements of the surface pressure exerted by the orthosis on the limb 110 are carried out using the interface pressure sensors 141-143;
merging of the different data and correlation in step 407, analysis of the measurements carried out in order, in particular, to determine the effectiveness and the impact of the compression orthosis on the vascular system of said limb 110 and, finally, selecting or adapting an orthosis in a specific manner;
optionally, assisting the prescription of particular compression orthoses in step 408, resulting from the previous measurements and analyses.

FIG. 5 shows an analysis sequence of ultrasound images produced during the step of biomechanical measurements,

According to this particular analysis method, a region of interest (ROI) is first determined 501. It comprises, in particular, the vascular vessel 511, the morphological characteristics of which are sought.

Then the region of interest is binarized in step 502 as a function of a threshold defined as a function of the parameters of measurements and/or of the user; it can for example be carried out according to a method called gradient calculation, making it possible to carry out adaptive thresholding. It can also be predefined in a manner that is invariant with respect to the images and/or the patients.

The following step 503 consists of reconstructing a coherent geometry of the cell thus isolated in the region of interest, via an operation of mathematical morphology.

It is then possible to determine the position of the walls of the vessel in step 504 and in step 505. According to the orientation of these walls and with respect to the vicinity of the central part of the region of interest, the average diameter of the vessel is calculated. The position and evolution of the cross-section along the blood vessel is measured.

Advantageously, the position, the orientation and the dimensions of the walls of the vessel are measured—optionally using a simplified ellipsoidal model of the cross-section of said vessel, in order to calculate the transverse surface (and evolution thereof) of said vessel at least one position.

At least some of the diameters and/or positions and/or dimensions and/or orientations calculated are recorded in a file.

A simplified visualization 506—in the form of an ellipsoidal representation of the vessels—makes it possible to observe in real time the variation in the diameter of said vessels, said variation being calculated according to a longitudinal and/or transverse section.

Of course, the invention is not limited to the examples which have just been described and numerous adjustments can be made to these examples without exceeding the scope of the invention. In particular, the different characteristics, forms, variants and embodiments of the invention can be combined together in various combinations to the extent that they are not incompatible or mutually exclusive. In particular, all the variants and embodiments described previously can be combined.

1. A non-wounding system for bio-morphological characterization of a human limb comprising a device for geometric and volumetric measurements, comprising:
a plurality of systems for three-dimensional images acquisition arranged for imaging said limb;
an articulated and motorized frame arranged both for positioning at least some of the plurality of the systems for acquisition in a peripheral manner with respect to said limb, and also for moving at least some of the plurality of the systems for acquisition with respect to said limb;
a device for processing the geometric and volumetric data, arranged for representing the acquisition data in the form of a plurality of points presenting a set of coordinates in a three-dimensional frame of reference;
a device for anatomical and biomechanical measurements comprising a probe holder comprising:
an ultrasound probe for imaging the vascular system relating to said limb;
a force sensor arranged for measuring the pressure exerted by said probe on the limb, said force sensor being fixed to said ultrasound probe; and
a device for analysis, arranged both for merging at least some of the volumetric data and at least some of the anatomical and biomechanical data, and also for determining morphological variables of the limb and/or biomechanical variables of the vascular system of said limb.

2. The system according to claim 1, characterized in that at least some of the plurality of systems for three-dimensional images acquisition of the system according to the invention operate synchronously.

3. The system according to claim 1, characterized in that the device for geometric and volumetric measurements also comprises a tool assisting the geometrical and volumetric measurement of said limb, arranged for determining representative areas of the limb for the determination of its shape and its volume.

4. The system according to claim 1, characterized in that the probe holder is mounted on an articulated and/or motorized arm fixed to the frame, and arranged for bringing said probe holder into contact with the limb and/or for moving said probe holder on said limb.

5. The system according to claim 1, characterized in that the device for anatomical and biomechanical measurements comprises at least one sensor for measuring the interface pressure, said at least one sensor being placed in contact with the skin of said limb.

6. A method for assisting the definition, selection or adaptation of a compression orthosis for a limb, implementing the system for bio-morphological characterization according to claim 1, comprising at least one of the following steps:
geometric and volumetric measurements of said limb;
biomechanical measurements of said limb;
merging of the geometric and/or volumetric and biomechanical measurements in order to correlate at least some of said geometric and/or volumetric measurements and at least some of said biomechanical measurements; and
determining at least one biometric variable and/or at least one volumetric parameter.

7. The method according to claim 6, characterized in that the step of biomechanical measurements of the limb is carried out at least during the step of geometric and/or volumetric measurements.

8. The method according to claim 6, characterized in that it also comprises a step of defining, selecting or adapting a compression orthosis for the limb, as a function of the at least one biometric variable and/or the at least one geometric and/or volumetric variable.

9. The method according to claim 6, characterized in that it comprises an additional step of developing a biomechanical model predicting the effects of the compression orthosis on the limb and its vascular system.
